(19) United States
(12) Patent Application Publication
(10) Pub. No.: US 2020/0383577 A1
(43) Pub. Date: Dec. 10, 2020

(54) SYSTEMS, DEVICE AND METHODS PROVIDING A COMBINED ANALYSIS OF IMAGING AND LASER MEASUREMENT

(71) Applicant: LEIBNIZ-INSTITUT FÜR PHOTONISCHE TECHNOLOGIEN E.V., Jena (DE)

(72) Inventors: IWAN W. SCHIE, Jena (DE); WEI YANG, Jena (DE); JÜRGEN POPP, Jena-Kunitz (DE)

(21) Appl. No.: 16/769,469

(22) PCT Filed: Dec. 12, 2018
(86) PCT No.: PCT/EP2018/084493

§ 371 Date: Jun. 3, 2020

(30) Foreign Application Priority Data
Dec. 13, 2017 (EP) 10 2017 129 837.1

(51) Int. Cl. A61B 5/00; G01J 3/44; A61B 5/06

(52) U.S. Cl. CPC A61B 5/0075; G01J 3/44; A61B 18/20; A61B 5/061; A61B 5/0084; A61B 5/0077

(57) ABSTRACT

Exemplary apparatus, device, system and method can be provided for examining a sample, which can comprising and/or utilize an imaging device for obtaining an overview image of the sample. A measuring instrument can also be used for locally interrogating at least one property of the sample with a laser beam which emerges from an aperture. Additionally, a tracking arrangement/system/device can be utilized for determining the location on the sample which is currently being interrogated with the laser beam. Additionally, memory or any other electronic storage device can be utilized, in which the property interrogated with the laser beam can be associated with the determined location on the sample. For example, the tracking arrangement/system/device can be designed and/or configured to determine the location at which the laser beam impacts or strikes the sample by evaluating the laser spot produced thereby from the overview image, and/or to determine this location by measuring the position and orientation of the aperture.

### CROSS REFERENCE TO RELATED APPLICATION(S)

This application relates to, and claims the benefit and priority from International Patent Application No. PCT/EP2018/084493 filed on Dec. 12, 2018 that published as International Patent Publication No. WO 2019/115589 on Jun. 20, 2019, which claims the benefit and priority from German Patent Application No. 10 2017 129 837.1 filed on Dec. 13, 2017, the entire disclosures of which are incorporated herein by reference in their entireties.

### FIELD OF THE DISCLOSURE

The present disclosure relates to devices, systems and methods which facilitate a fusion of information obtained over a large area by imaging of a sample with locally requested information regarding certain properties of the sample.

### BACKGROUND INFORMATION

Imaging techniques are often used to investigate properties of samples, such as, e.g., biological tissue. Optical images, in particular, can be obtained quickly, even on spatially extended samples, such as, e.g., a complete organ. For example, such images can contain the information which, part of the spectrum offered by a light source, can be reflected by the sample.

At times, this information may not be sufficient for information requested at the beginning of the investigation. For example, an optical change in tissue may be due to a disease, although it may also have another cause. A chemical examination, for example with molecule-specific Raman spectroscopy, can clarify whether tumour markers are present in the tissue.

However, such investigation can be difficult to perform in vivo. Most instruments for an investigation using a Raman spectroscopy can be designed for the analysis of small samples on slides or in cuvettes, which would first likely have to be removed from an extended organ. Examples of such devices are optical microscopes extended by a Raman spectrometer. In principle, the laser beam can be directed at the organ from a greater distance. However, it may then be difficult to collect enough of the Raman-scattered light emitted in all directions.

Thus, there may be a need to address and/or resolve at least some of the deficiencies and/or issues present in the prior devices, systems and/or methods.

### EXEMPLARY OBJECTS OF THE PRESENT DISCLOSURE

It is therefore one of the objects of the present disclosure to provide device, system and method using which a property can be locally interrogated with a laser beam on a spatially extended sample. In this exemplary manner, the signal-to-noise ratio can be improved as compared to the conventional devices, systems and method, and the information can thus obtained can also be fused or otherwise combined with information obtained over a large area by imaging. This exemplary object and/or task can be facilitated with exemplary apparatus, devices, systems and methods according to various exemplary embodiments of the present disclosure.

### SUMMARY OF EXEMPLARY EMBODIMENTS

Thus, according to various exemplary embodiments of the present disclosure, devices, system and methods can be provided for analyzing or examining at least one sample. The exemplary device, system and method can comprises and/or utilize an imaging device for obtaining an overview image of the sample, such as a camera or an array of cameras. The exemplary device, system and method can further comprises and/or utilize a measuring instrument for locally interrogating at least one property of the sample with a laser beam which can emerge or extend from an aperture, a tracking arrangement/device/system configured to determine the location on the sample which is currently interrogated with the laser beam, and memory or other electronic arrangement in which the property interrogated with the laser beam is associated with the determined location on the sample.

In accordance with various exemplary embodiments of the present disclosure, the tracking arrangement/device/system can be configured to determine the location on the sample at which the laser beam impinges on the sample by, e.g., evaluating the laser point thus produced from the overview image, and/or to determine this location by measuring the position and orientation of the aperture.

In this exemplary manner, it is possible to get close to the sample with the aperture even in the case of a spatially extended sample, such as a complete organ. Thus, the aperture can cover a large part of the spatial angle into which the sample itself emits light when interrogated with the laser beam. For example, a large portion of this light can then be collected and used for evaluation.

This represents a significant and notable paradigm shift compared to conventional microscopes, which are extended by Raman spectrometers. With these microscopes, the location on the sample to be examined with the laser beam is known in advance, since the sample is mounted on a positioning table, for example, which is moved in a defined manner relative to the aperture. This can make it less difficult to assign the property scanned with the laser beam to a specific location on the sample. However, it was recognized that with spatially extended samples, it can be technically difficult to move the aperture close to the sample in a known manner from a great distance. Instead, it may be advantageous to initially completely ignore or avoid the knowledge of which location on the sample is currently being scanned with the laser beam and to determine such information again at a later point in time.

However, the concept of querying a property on the sample with the laser beam may not be limited to the fact that the sample generates an optical signal in response to the laser beam and this signal is evaluated. For example, the sample can also be heated locally by the laser beam and this heating can be observed in the far field with a thermal imaging camera. Likewise, material can be removed locally from the sample and sucked into a mass spectrometer to determine its chemical composition.

In an exemplary beneficial configuration according to an exemplary embodiment of the present disclosure, the aperture can be part of a probe which can be manually guided to the sample by the operator of the device. While the aim can usually be to automate and mechanize as many steps as possible, manual positioning of the aperture is particularly advantageous when examining tissue during an operation. A mechanized positioning with a robot arm would require considerable effort in this case, whereby the safety would have to be ensured in particular that no injuries are caused by excessive force of the robot arm. A significant amount of space may also be needed for the robot arm, which is often not available in an operation scenario. By carrying out the positioning manually and shifting the automation to the subsequent determination of the examined location on the sample, the strengths of the operator on the one hand and the technology on the other hand can be optimally combined. This also makes it possible to realize a transportable unit that is not bound to a specific location.

The exemplary configuration of manual manoeuvrability is not limited to the fact that the probe is held in the hand. Rather, this term also covers, for example, the fact that the probe is guided to an organ through the working channel of an endoscope.

In general, due to time constraints, the laser beam is not used to examine the entire extended sample in detail, but rather certain locations are selected for this examination based on the overview image. For example, the operator can use the fast hand-eye coordination and at the same time use his sense of touch to avoid injuries due to excessive force applied to tissue. The operator can thus concentrate purely on the medical aspects of the examination, while the device in the background takes care of the complexity of merging the result of the detailed examination by the laser beam with the overview image.

According to an exemplary embodiment of the present disclosure, the probe can have a manually operated trigger for the query of the property by the measuring instrument. It is not possible to query the property in real time for every type of examination. For example, the recording of a Raman spectrum can take a particular amount of time, e.g., several seconds. This can mean that after positioning the probe at a point of interest, it may be important to wait for the end of the current recording. If, on the other hand, the recording can be started by pressing the trigger, this waiting time is not necessary.

If the distance between the probe and the sample changes, the focal plane of the laser beam exiting the aperture may also change. To what extent this affects the interrogation of the property of the sample with the laser beam depends on the physical contrast mechanism used in the interrogation and the speed of the interrogation. For example, the acquisition of a Raman spectrum can take a few seconds and become “blurred” if there is too much relative movement between the probe and the sample, similar to a photograph taken with a correspondingly long exposure time. The relative movement can be caused, for example, by manual guidance of the probe, but also, for example, by the natural movement of the sample, which may be a living organ. Therefore, in another particularly advantageous configuration of the present disclosure, tracking means are provided which are designed to track the focal plane of the laser beam emerging from the aperture to a change in the distance between the aperture and the sample.

Such exemplary tracking device, system/apparatus can be configured, for example, to automatically readjust the focal plane of the laser and/or the focal plane of the light coupling into an optical waveguide leading to the aperture. For this exemplary purpose, the distance between the probe and the sample can be determined in any way. For example, the probe can have a measuring device for the distance between the probe and the sample. This measuring device can, for example, have a transmitter for an electromagnetic wave and/or for ultrasound, and a receiver for the wave reflected by the sample or the ultrasound reflected by the sample. However, the distance between the aperture and the sample can also be determined, for example, from any image showing both the probe and the sample. This image can be or include a camera image, for example, and can also be obtained in any other way and/or, for example, by evaluating terahertz radiation.

The refocusing can be performed, for example, using exemplary fast mechanical traversing mechanisms, with the aid of liquid lenses or with any other adaptive optics.

Tracking of the focal plane can also facilitate maintaining the focal plane stable when the aperture is moved, for example manually, over an extended area on the sample. As a result, the measured values obtained at different locations on the sample become more comparable for the property of the sample.

In an exemplary embodiment of the present disclosure, the tracking system/device/arrangement can comprise image evaluation logic configured to identify a location where the luminance of the overview image exceeds a threshold value, a center of gravity of the luminance of the overview image, and/or a location where a spatial profile of the luminance of the overview image matches the beam profile of the laser beam, as the location on the sample currently being scanned by the laser beam. The laser light can be much more strongly directed than, for example, lamp light, with which an optical overview image is produced, and is thus typically dominant in luminance. A reliable identification of the location currently scanned with the laser beam can also be facilitated if parts of the sample have the same color as the laser beam.

In another advantageous configuration according to a further exemplary embodiment of the present disclosure, a modulator can be provided that is configured for modlating the laser beam with a frequency w is provided. Furthermore, the exemplary device can be provided configured to obtain the overview image is extended in such a way that it is capable of recording a time sequence of overview images. The tracking system/device./arrangement can comprise an image evaluation logic which is designed to identify from the temporal sequence of overview images a location on the sample where the luminance is modulated with the frequency can be the location on the sample which is currently being scanned with the laser beam. In this exemplary manner, the currently scanned location can still be identified even if the laser intensity used is very low and the luminance caused by the laser beam lags behind the dominance caused by other light sources.

In another exemplary advantageous configuration according to an exemplary embodiment of the present disclosure, the tracking device/system/arrangement can comprise at least two laser scanners or radio transmitters configured to spatially track the aperture or the probe. These exemplary devices may, for example, be placed in the corners of an operating theatre, where they do not interfere, and track the position and orientation of the probe within the whole operating theatre.

In another exemplary configuration according to an exemplary embodiment of the present disclosure, the measuring instrument can additionally comprise a scanning device designed to change the angle at which the laser beam exits the aperture. In this way, the point-like examination with the laser beam can be extended to examine a limited area on the sample.

In an additional exemplary configuration according to an exemplary embodiment of the present disclosure, the measuring instrument can include a Raman spectrometer configured to query the chemical composition of the sample. Each molecule can leave a characteristic fingerprint in the Raman spectrum, so that, for example, the composition of mixtures can also be clearly determined. In particular, e.g., tumor tissue can be clearly identified by the presence of tumor markers. Furthermore, the light scattered by the sample Raman-scattered light can be separated by spectral filtering from the laser beam used for scanning, since it is wavelength-shifted from the laser beam.

Any desired correction can be applied to the Raman spectrum, according to an exemplary embodiment of the present disclosure. For example, a fluorescent background can be separated by a fit using polynomials, EMSC or a least squares method.

In a further exemplary configuration according to an exemplary embodiment of the present disclosure, an excitation laser on the one hand and the Raman spectrometer on the other hand can be connected via a fiber coupler, whose division ratio is wavelength-dependent, to a common optical fiber leading to the aperture. For this exemplary purpose, the fiber coupler may contain, for example, a dichroic mirror. In this way, even greater distances of several meters between the excitation laser, the spectrometer and the sample can be covered. The excitation laser and the spectrometer then, e.g., do not need to occupy space in the operating room, for example, in the immediate vicinity of the operating field, and can be located in a place where they do not interfere. The optical fiber with the probe can be guided to the operator of the device in any way, for example from the ceiling of the operating theatre, in order to avoid tripping hazards.

In another particularly advantageous configuration of the present disclosure, an output unit is provided which is designed to superimpose a representation of the property stored in the memory and scanned by the laser beam on the overview image of the sample at the location associated by the memory. In this way, the overview image is upgraded to an “augmented reality” in a way which is directly visible to the operator.

For example, a Raman spectrum can be evaluated to search for a given canon of chemical substances. For example, a linear combination of Raman spectra of substances from this canon can be fitted to the recorded Raman spectrum in such a way that a maximum match is obtained. The coefficients of the linear combination can then provide information about the proportions in which the sought-after substances are present at the sought-after location. In such manner, for example, each substance from the canon can be assigned a color, and these colors can be applied, for example, in the representation with intensities determined by the coefficients of the linear combination.

The canon of substances to be searched for is freely selectable and can be dynamically adjusted, especially by the operator. For example, it is possible to hide certain substances for the sake of clarity.

In another exemplary configuration according to an exemplary embodiment of the present disclosure, a projector can be provided which is designed or configured to project onto the sample a representation of the property stored in the memory and scanned by the laser beam at the location associated by the memory. With this, e.g., “augmented reality” can be further refined to the extent that the operator no longer needs to move his gaze back and forth between the sample, for example the organ, and a computer screen. For example, the operator can move the probe over an area of the organ whose chemical composition interests him, and “draw” the chemical composition determined by Raman spectroscopy directly onto the organ itself. Projection is particularly advantageous during longer operations, as each change of view between the organ and a computer screen requires the eyes to be adjusted to a different distance. These changes can become tiring over time.

In yet another exemplary configuration according to an exemplary embodiment of the present disclosure, the imaging device comprises a bright field camera. The captured overview image then corresponds to the normal way of seeing in humans. In this respect, it is therefore would not be necessary for the operator to get used to moving his gaze back and forth between the sample and a computer screen displaying the overview image.

In another exemplary configuration according to an exemplary embodiment of the present disclosure, the imaging device can be designed or configured to take a three-dimensional overview image of the sample. A three-dimensional overview image can be used in particular to control a projector to project the representation of the queried property onto the sample at the correct location associated in memory. The imaging device may, e.g., comprise a stereo camera and/or a strip photometric device.

For example, the local angle of incidence of the illumination on the sample, and thus the local orientation of the sample surface, can be determined by a least-squares fit of overview images taken under illumination from different directions. The orientation of the sample surface can then be determined that leads to an intensity distribution that is least contradictory to the intensity distributions actually observed.

In a further exemplary embodiment according to an exemplary embodiment of the present disclosure, a switching device can be provided which is designed or configured to switch the intensity of the laser beam between a first, lower level for sensing the property of the sample and a second, higher level for removing material from the sample, and/or for changing material of the sample. This switching device can be a shutter or a Pockels cell, for example. The device can then also be used to change the property scanned by the laser beam. For example, a chemical contaminant or tumor tissue can be ablated.

The exemplary laser used for ablation and/or modification of material need not be the same as the laser used for interrogation. The laser intensity can also be switched by releasing the beam path to the sample from a second laser with higher intensity. For example, the laser used for scanning can be a continuous wave laser, while the laser used for ablation and/or modification emits ultrashort pulses of very high intensity. Such pulses can interact directly with the electron shells of atoms of the material to be ablated. The material can then be ablated without heating the surrounding area on the sample to a large extent.

For example, the switching device may be controlled by a manually operated trigger mounted on a manually guided probe. The operator can then, for example, use the probe as a “chemical eraser” using the “Augmented Reality” described above to directly “erase” detected undesirable substances or tissue changes by wiping the probe across them.

In this context, although the use of the device to assist surgery is an essential “use case”, it is not limited to this. For example, a component consisting of a metal alloy or a mixture of plastics can also be examined with a manually guided probe to determine whether the composition of the alloy or mixture is homogeneous over the entire component and whether the component thus has the use properties promised by this composition through and through. In the same way, for example, a weld seam or an adhesive joint can be examined to determine whether the respective properties are homogeneous. In the exemplary case of a glued joint, for example, areas can be identified in which the adhesive has not reacted through to its hardened form. Possible sources of error here are, for example, insufficient lighting in the case of a light-activated adhesive or insufficient mixing of the components of a multi-component adhesive.

In still another exemplary configuration according to an exemplary embodiment of the present disclosure, the switching device can be connected to the measuring device so that it is automatically triggered when the property scanned with the laser beam fulfils a predetermined condition. For example, the switching device can be automatically activated if a certain substance has been identified at the location scanned with the laser beam. The switching device can thus be activated by an automatic trigger mechanism triggered by the presence of a certain chemical substance. The presence of the chemical substance can be detected, for example, by Raman spectroscopy, but also, for example, by the emission of fluorescent light in response to the laser beam used for interrogation. This allows the user to perform chemically controlled ablation.

Chemically controlled ablation is not only useful in the medical field. It can also be used in the cosmetic field, for example, to selectively chemically transform or split up tattoo colours without leaving scars on the treated skin. Furthermore, graffiti colours can also be selectively removed from surfaces that are too sensitive for the application of chemical solvents.

### BRIEF DESCRIPTION OF THE DRAWINGS

Further exemplary embodiments of the present disclosure are detailed in the description of the Figures, where this description shall not limit the scope of the exemplary embodiments of the present disclosure. The Figures show:

FIG. 1 is an exemplary on-scaled diagram of an exemplary system of the construction of a fixture according to an exemplary embodiment of the present disclosure;

FIG. 2a is a diagram of an exemplary representation of “augmented reality” on an output unit, according to an exemplary embodiment of the present disclosure;

FIG. 2b is a diagram of an exemplary representation of “augmented reality” on a sample, according to an exemplary embodiment of the present disclosure;

FIG. 3 is a diagram of a system providing switching between an excitation laser for querying and/or determining a property of sample and an ablation laser for removing material from the sample, according to an exemplary embodiment of the present disclosure; and

FIGS. 4a-4c are diagrams of a superposition of optical and chemical information using the example of two workpieces, which are glued together with a glue joint, according to an exemplary embodiment of the present disclosure.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative embodiments. It is intended that changes and modifications can be made to the described embodiments without departing from the true scope and spirit of the subject disclosure as defined by the appended claims.

### DETAILED DESCRIPTION OF EXEMPLARY EMBODIMENTS

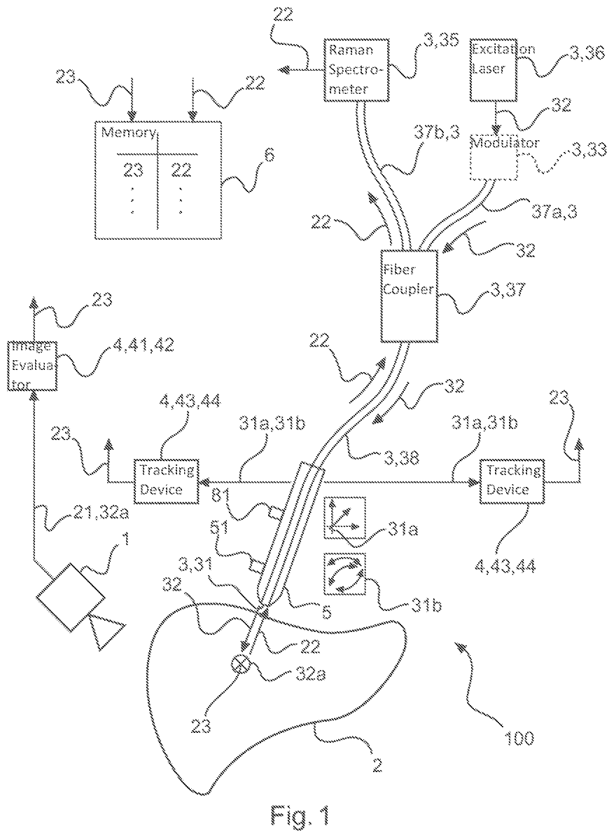

FIG. 1 shows a schematic exemplary on-scaled diagram of an apparatus 100, according to an exemplary embodiment of the present disclosure. In this example, sample 2 to be examined is a liver in vivo. It should be understood that other organs and body parts are omitted for the sake of clarity, and are within the scope of the present disclosure.

A probe 5 can be guided over sample 2 and/or by the operator of the apparatus 100. A glass fiber 38 can be passed through the probe 5, which terminates in an aperture 31. An excitation laser 36 can emit a laser beam 32, which can be optionally modulated with a frequency w in a modulator 33. With a first glass fiber 37a, the laser beam 32 passes into a fiber coupler 37 with wavelength-dependent splitting ratio. The laser beam 32 emerges from the aperture 31, and generates/produces a laser spot 32a at location 23 on sample 2. At this exemplary location 23, Raman-scattered light is generated, which is characteristic of the local chemical composition of sample 2 as the interrogated property 22. The Raman-scattered light, symbolized by the reference sign 22 for the information contained in it, can be guided through the fiber coupler 37 into a second glass fiber 37b, which leads to a Raman spectrometer 35. The exemplary distance between the aperture 31 and the sample 2 is shown in FIG. 1 for the sake of clarity. The Raman spectrometer 35 can determine the local chemical composition 22 of sample 2 at location 23, although it has no knowledge of where this location 23 is located on the sample 2.

For example, FIG. 1 illustrates two exemplary ways to determine the location 23. A tracking device 4 for this purpose may comprise at least two laser scanners 43 or radio transmitters 44 which determine the position 31a and the orientation 31b of the probe 5, and thus also the aperture 31. The tracking device 4 may alternatively or in combination also comprise an image evaluation logic 41, 42 which contains the overview image 21 of probe 2 supplied by a camera 1 or another optical or electronic visualization/image/video capture device, including the laser point 32a generated therein by the laser beam 32, and evaluates the position of the laser point 32a as the location 23 from the overview image 21.

Each location 23 can be stored in a memory 6 (or in another electronic storage device), e.g., together with the associated queried property 22.

The aperture 31, the excitation laser 36, the modulator 33, the fiber coupler 37, the optical fibers 37, 37a and 38 connected to the fiber coupler 37 and/or the Raman spectrometer 35 together form the measuring instrument 3 for querying the property 22 of the sample 2.

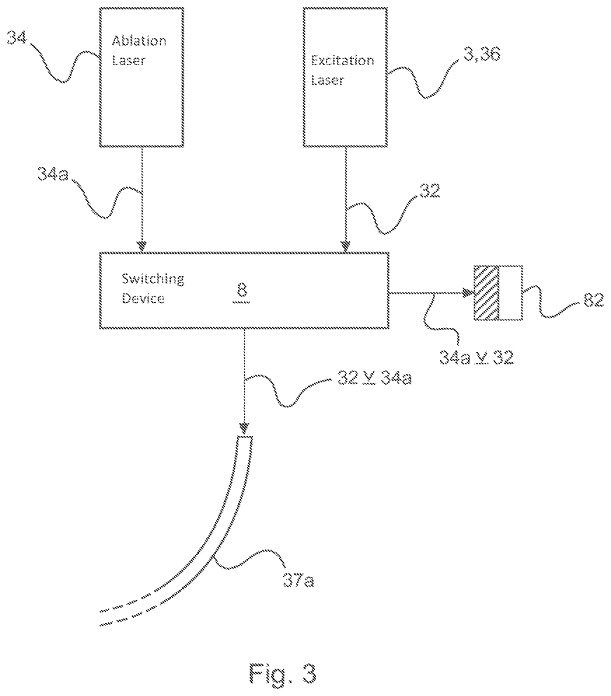

Probe 5 can contain a first trigger 51, with which the operator of the device 100 can trigger the recording of a Raman spectrum by the Raman spectrometer 35. Probe 5 can also contain a second trigger 81, which can be used to trigger the switching device 8 for material removal, which is explained in detail herein with reference to FIG. 3. The signal connections of the triggers 51 and 81 are not shown in FIG. 1 for the sake of clarity.

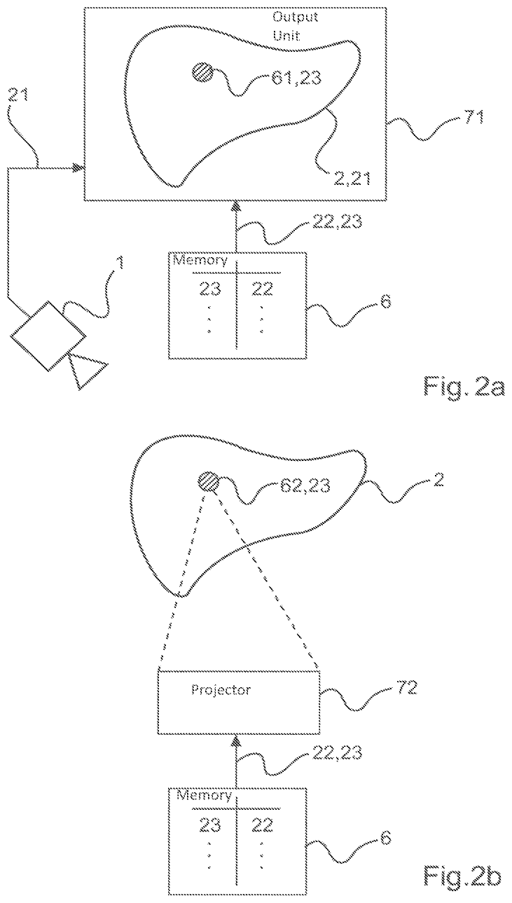

FIG. 2a shows an exemplary diagram of a use of an “augmented reality” that can be displayed on an output unit 71 using the information stored in, e.g., memory 6 or in another electronic storage device, according to an exemplary embodiment of the present disclosure. For example, the output unit 71 can receive the overview image 21 of sample 2 from camera 1 and displays it in the background. According to an exemplary embodiment of the present disclosure, at the same time, the output unit 71 can receive from memory 6 or from another electronic storage device the values of the queried property 22 together with the respective locations 23 on sample 2. Based on such exemplary performance and/or results, the output unit 71 can determine a representation 61 in which, for example, different chemical substances are displayed in different colors. The representation 61 can be superimposed on the overview representation 21 of sample 2.

FIG. 2b illustrates an exemplary diagram an “Augmented Reality” creation that does not require a turn of gaze away from Sample 2 and towards an Output Unit 71, according to an exemplary embodiment of the present disclosure. For example, a projector 72 can generate a representation 62 from the information stored in memory 6, which can be color-coded, for example, analogous to representation 61 according to FIG. 2a. In contrast to FIG. 2a, the exemplary representation of FIG. 2B can be projected directly onto the sample 2. Thus, each value for the queried property 22 is projected onto the corresponding location 23.

FIG. 3 shows an exemplary diagram of switching between a query for property 22 and material removal from the sample 2, according to an exemplary embodiment of the present disclosure. For example, the continuous laser beam 32 of the excitation laser 36 and the pulsed laser beam 34a of the ablation laser 34 can each be guided into the switching device 8. The switching device 8 couples exactly one of the beams 32 and 34a each into the first optical fiber 37a, which leads to the fiber coupler 37 as shown in FIG. 1. The other beam can be directed to a beam dump 82 where it is converted into heat. In this exemplary manner, the lasers 36 and 34 themselves do not have to be constantly switched on and off, which would be bad for their service life.

FIGS. 4a-4C illustrate exemplary diagrams of other exemplary applications in which normal optical contrast and chemical contrast can be combined using the device 100, according to various exemplary embodiments of the present disclosure. For example, the sample 2, an arrangement of a first workpiece 91 and a second workpiece 92, which are glued together by a glue joint 93, can be examined using such exemplary embodiments.

In particular, FIG. 4a shows an exemplary diagram of those features of the sample 2 which are visible in a normal optical overview image 21. The first workpiece 91 has substantially horizontal grooves 91a and the second workpiece 92 has substantially vertical grooves 92a. The scoring marks 91a, 92a were each created during the manufacture of workpieces 91 and 92. The adhesive joint 93 appears colourless and without any special structure.

FIG. 4b shows an exemplary diagram in which sample 2 has already been partially examined with probe 5. The adhesive joint 93 is examined successively from left to right. Where the probe 5 has already been, it was identified that the adhesive joint 93 consists of properly cured adhesive 93a. This information can be output on an output unit 71 as explained in FIG. 2a or, for example, projected directly onto sample 2 as explained in FIG. 2b.

FIG. 4c illustrates an exemplary diagram of the condition in which the complete adhesive joint 93 has been scanned with probe 5. In the area of the adhesive joint 93 not yet examined in FIG. 4b, it is now apparent that the first component 93b and the second component 93c of the adhesive are present in separate phases and have not reacted to produce the final shape 93a. In this exemplary area, the adhesive joint 93 is therefore faulty and not loadable.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY LIST OF REFERENCE SIGNS

1 Device/arrangement configured to obtain the overview image 21
2 Sample
21 Overview image of sample 2
22 Property of sample 2 interrogated with laser beam 32
23 Location on sample 2, where laser beam 32 interrogates property 22
3 Measuring instrument for local interrogating of the property
31 Aperture for laser beam 3
31a Position of the aperture 31
31b Orientation of aperture 31 in space
32 Laser beam
32a Laser spot generated by laser beam 32 on sample 2
33 Modulator for laser beam 32
34 Ablation laser
34a Beam of the ablation laser 34
35 Raman spectrometer
36 Excitation laser
37 Fiber coupler
37a Input of the fiber coupler 37 for laser beams 32, 34a
37b Output of the fiber coupler 37 for interrogated property 22
38 Common optical fiber for laser beam 32, 34a and property 22
4 Tracking Arrangement/system/device configured to track location 23 on trial 2
41, 42 Image evaluation logic
43 Laser scanner
44 Radio transmitter
5 Probe
51 Trigger which can cause the interrogation of the property 22 when operated
6 Memory that associates property 22 with locations 23
61, 62 Representations of information in memory 6
71 Output device provided for overview screen 21 and representation 61
72 Projector for presentation 62 on trial 2
8 Switching device
81 Release for switching device
82 Beam dump
91 First workpiece
91a Scoring in first workpiece 91
92 Second workpiece
92a Scoring in second workpiece 92
93 Glued joint between workpieces 91 and 92
93a Fully cured adhesive
93b First component of the adhesive
93c Second component of the adhesive
100 Apparatus

1-17. (canceled)

18. An apparatus for analyzing a sample, comprising:
an imaging device configured to obtain at least one overview image of the sample;
a measuring instrument configured to locally interrogate at least one property of the sample using a laser beam which emerges from an aperture;
a tracking device configured to track and determine a location on the sample which is currently being interrogated using the laser beam; and
an electronic storage device configured to store the at least one property interrogated using the laser beam,
wherein the stored at least one property is associated with the determined location on the sample, and wherein the tracking device is further designed and configured to determine the location at which the laser beam strikes or impacts the sample as the location on the sample which is currently being interrogated using the laser beam, by at least one of:
(i) evaluating a laser spot produced from the at least one overview image, or
(ii) measuring a position and an orientation of the aperture.

19. The apparatus according to claim 18, wherein the aperture is part of a probe which is manually guidable to the sample by an operator of the apparatus.

20. The apparatus according to claim 19, wherein the probe includes a manually operable trigger, and wherein the measuring instrument is configured to interrogate the at least one property in response to operation of the trigger.

21. The apparatus according to claim 18, wherein the tracking device is further designed and configured to track a focal plane of the laser beam emerging from the aperture with respect to a change in the distance between the aperture and the sample.

22. The apparatus according to claim 18, wherein the tracking device further comprises an image evaluation logic system which is designed and configured to (i) detect a location where a luminance of the at least one overview image exceeds a threshold value, and (ii) identify at least one of a center of the luminance of the at least one overview image or a location where a spatial profile of the luminance of the at least one overview image matches the beam profile of the laser beam as the location on the sample which is currently being interrogated with the laser beam.

23. The apparatus according to claim 18, further comprising a modulator configured to effectuate a modulation of an intensity of the laser beam with a frequency ω,
wherein the at least one overview image comprises a plurality of overview images,
wherein the imaging device is designed and configured to obtain a time sequence of the plurality of overview images, and
wherein the tracking device comprise an image evaluation logic system which is designed and configured to identify, from the time sequence of the plurality of overview images, a location where the luminance is modulated with the frequency ω as the location on the sample which is currently being interrogated with the laser beam.

24. The apparatus according to claim 18, wherein the tracking device comprises at least two laser scanners or at least two radio transmitters configured to spatially track the aperture or a probe enclosing the aperture, respectively.

25. The apparatus according to claim 18, wherein the measuring instrument comprises a scanning device which is designed and configured to modify or control an angle of an exit of the laser beam from the aperture.

26. The apparatus according to claim 18, wherein the measuring instrument comprises a Raman spectrometer configured to interrogate or analyze at least one chemical composition of the sample.

27. The apparatus according to claim 26, wherein the laser beam is generated by an excitation laser, wherein the excitation laser and the Raman spectrometer are connected via a fiber coupler to a glass fiber leading to the aperture, and wherein the fiber coupler has a division ratio that is wavelength-dependent.

28. The apparatus according to claim 18, further comprising an output unit designed and configured to superimpose a representation of the at least one property stored in the electronic storage device and scanned with the laser beam on the overview image of the sample at the location on the sample which is currently being interrogated using the laser beam.

29. The apparatus according to claim 18, further comprising a projector designed and configured to project a representation of the at least one property which is stored in the in the electronic storage device and interrogated with the laser beam onto the sample at the location of the sample which is currently being interrogated using the laser beam.

30. The apparatus according to claim 18, wherein the imaging device comprises a bright field camera.

31. The apparatus according to claim 18, wherein the at least one overview image includes a three-dimensional overview image of the sample, and wherein the imaging device is designed and configured to obtain the three-dimensional overview image of the sample.

32. The apparatus according to claim 31, wherein the imaging device comprises at least one of a stereo camera or a strip photometry device.

33. The apparatus according to claim 18, further comprising a switching device designed and configured to switch an intensity of the laser beam between (i) a first lower level so as to interrogate the at least one property of the sample, and (ii) a second higher level so as to at least one of remove at least one material from the sample or change the at least one material of the sample.

34. The apparatus according to claim 33, wherein the switching device is connected to the measuring instrument so that the switching device is automatically triggered when the at least one property scanned with the laser beam fulfils a predetermined condition.

35. A method for analyzing a sample, comprising:
obtaining at least one overview image of the sample,
locally interrogating at least one property of the sample using a laser beam which emerges from an aperture;
tracking and determining a location on the sample which is currently being interrogated using the laser beam;
electronically storing the at least one property interrogated using the laser beam, wherein the stored at least one property is associated with the determined location on the sample; and
determining the location at which the laser beam strikes or impacts the sample as the location on the sample which is currently being interrogated using the laser beam, by at least one of:
(i) evaluating a laser spot produced from the at least one overview image, or
(ii) measuring a position and an orientation of the aperture.
